(19) United States
(12) Patent Application Publication
(10) Pub. No.: US 2016/0030768 A1
(43) Pub. Date: Feb. 4, 2016

(54) System and Method for Improved High Dose Radiation Therapy Treatment Planning

(71) Applicant: The Regents of the University of California, Oakland, CA (US)

(72) Inventors: Daniel B. Ennis, Manhattan Beach, CA (US); Jeff Demanes, Los Angeles, CA (US); Subashini Srinivasan, Los Angeles, CA (US); Deborah Langman, Los Angeles, CA (US)

(21) Appl. No.: 14/777,094

(22) PCT Filed: Mar. 14, 2014
(86) PCT No.: PCT/US2014/029129

§ 371 Date: Sep. 15, 2015

Related U.S. Application Data
(60) Provisional application No. 61/791,848, filed on Mar. 15, 2013.

(51) Int. Cl. A61N 5/10; A61L 29/14; A61M 25/01

(52) U.S. Cl. CPC A61N 5/1039; A61N 5/1007; A61M 25/0127; A61L 29/14; A61M 2025/0004

(57) ABSTRACT

The present invention includes a catheter for providing contrast under magnetic resonance imaging (MRI). The catheter includes an elongated tubular member having a proximal end, a distal end and lumen. The catheter further includes a solution of saline and at least one contrast agent, where the solution is sealed within at least a portion of the catheter lumen.

### CROSS-REFERENCE TO RELATED APPLICATIONS

This application claims priority to U.S. Provisional Application No. 61/791,848 filed on Mar. 15, 2013, the contents of which are incorporated by reference herein in their entirety.

### BACKGROUND OF THE INVENTION

Brachytherapy is a method of radiation therapy that involves the placement of a source of radiation onto or directly into a tumor. The procedure consists of insertion of a hollow conduit (a catheter, needle or other applicator) by a physician (brachytherapist), followed by the temporary or permanent insertion of a radiation source into the target tissue. The applicator may be inserted into existing body cavities, where it is known as “intracavitary” or “intraluminal,” or it can be placed directly into the tissues, where it is termed “interstitial.” Interstitial implants are typically done with brachytherapy catheters or needles, which may generally be referred to as an applicator.

There are several types of brachytherapy, including high dose rate (HDR), low dose rate (LDR) and pulsed dose rate (PDR). Low dose rate brachytherapy is most commonly done as a permanent seed implant. In these types of brachytherapy, radioactive “seeds,” such as palladium or iodine, are permanently embedded into the target tissue. The dose is delivered as the sources decay to clinically insignificant levels of activity. In another format, LDR sources can be inserted and removed in a similar manner to HDR, except that the sources are left in place for several days while the patient is confined to the hospital.

High dose rate brachytherapy uses catheters or applicators similar to those used for LDR, however, the radiation source is high intensity and it is delivered with the use of a computerized robotic delivery device called a remote afterloader. The dose is typically given in less than one hour on multiple separate occasions rather than days (as in LDR), hence it is termed “high dose rate remote afterloading” or HDR for short. PDR is a hybrid approach that involves frequent (hourly, for example) insertion and removal of the radiation source throughout the day so that the radiation dose is given in bursts rather than continuously but treatment is delivered over a time course similar to LDR.

In practice, the applicator is inserted into the treatment site so that the distal region is within the treatment target and the proximal end of the applicator protrudes outside the body so that it can be connected to the afterloader. The tiny but powerful (5-10 Ci) radiation source attached to the distal end of a fine cable is robotically fed into the applicator to the treatment site to various specified “dwell” locations within the implanted applicator for prescribed amounts of time. Upon completion of the treatment cycle, the radiation source is retracted from the patient and back into the afterloader radiation safe.

Brachytherapy applicator devices (typically plastic catheters and various kinds of metal needs or tubes) are usually easy to identify and locate with computed tomography (CT) but they are difficult to visualize with magnetic resonance imaging (MRI). The patient anatomy and extent of the disease, however, are typically better defined on MRI. It is preferable in many cases to use the MRI system, which simultaneously demonstrates patient normal organ anatomy, tumor target, and the brachytherapy applicator, because it results in better and accurate radiation dosimetry and treatment delivery.

The existing state of the art reports on a device that is visualized well under both CT and MRI and can be found at (implantedmarkers dot com/ see: FusionCoil). Unfortunately, this device generates bright constrast on CT and dark contrast on MRI. Bright contrast under MRI, however, is preferred by clinicians (such as physicians, dosimetrists, and physicists) because all of the essential elements needed for treatment planning are apparent.

Thus, there is a need in the art for a system and method for obtaining both the visualization of tumor margin and bright contrast markers of the applicator's location on MRI. The present invention satisfies this need.

### SUMMARY OF THE INVENTION

The present invention relates to a catheter for providing contrast under magnetic resonance imaging (MRI). The catheter includes an elongated tubular member having a proximal end, a distal end and lumen, and a solution including saline and at least one contrast agent, wherein the solution is sealed within at least a portion of the lumen to form a solution pattern signature.

In one embodiment, the contrast agent is a gadolinium-containing contrast agent. In another embodiment, the contrast agent is gadobenate dimeglumine. In another embodiment, the contrast agent is gadopentetate dimeglumine. In another embodiment, the concentration of contrast agent is between 0.6-2.22 mmol/L. In another embodiment, the solution pattern signature comprises a variation in contrast agent concentration along a length of the tubular member. In another embodiment, the solution pattern signature comprises at least one interruption of the solution along a length of the tubular member, such that the solution is absent at the interruption. In another embodiment, the interruption comprises a gel. In another embodiment, the interruption comprises a bubble. In another embodiment, the interruption comprises a pinched region of the tubular member. In another embodiment, the interruption comprises a wall. In another embodiment, the elongated tubular member includes multiple compartments, wherein at least two of the compartments are isolated from each other by a wall within the lumen.

The present invention also relates to a method of visualizing a channel positioned in the tissue of a subject. The method includes the steps of positioning at least one channel in the tissue of a subject, inserting into each channel a catheter having a solution including saline and at least one contrast agent sealed within the lumen of the catheter, such that the solution forms a pattern signature, and imaging the inserted catheter by magnetic resonance imaging (MRI). In one embodiment, the channel is a high dose rate applicator (HDR) for brachytherapy. In another embodiment, the method further includes the step of constructing a treatment plan based on the visualization of each imaged catheter in each channel.

The present invention also relates to a system for visualizing a plurality of channels positioned in the tissue of a subject. The system includes a plurality of catheters for providing contrast under magnetic resonance imaging (MRI), each catheter comprising an elongated tubular member having a proximal end, a distal end and lumen, and a solution including saline and at least one contrast agent, wherein the solution is sealed within at least a portion of the lumen to form a pattern signature, wherein each catheter is uniquely identifiable by MR image and is associated with one of the one or more channels positioned in the tissue of the subject. In one embodiment, the one or more channels positioned in the tissue of the subject comprises one or more brachytherapy applicators. In another embodiment, the solution pattern signature comprises a variation in contrast agent concentration along a length of the tubular member. In another embodiment, the solution pattern signature comprises at least one interruption of the solution along a length of the tubular member, such that the solution is absent at the interruption. In another embodiment, the interruption comprises a gel. In another embodiment, the interruption comprises a bubble. In another embodiment, the interruption comprises a pinched region of the tubular member. In another embodiment, the interruption comprises a wall. In another embodiment, the elongated tubular member includes multiple compartments, wherein at least two of the compartments are isolated from each other by a wall within the lumen.

### BRIEF DESCRIPTION OF THE DRAWINGS

The following detailed description of preferred embodiments of the invention will be better understood when read in conjunction with the appended drawings. For the purpose of illustrating the invention, certain embodiments are shown in the drawings, which are presently preferred. It should be understood that the invention is not limited to the precise arrangements and instrumentalities of the embodiments as shown in the drawings.

FIG. 1 is a schematic of a catheter according to an aspect of the present invention. FIG. 1A depicts a side view of the catheter, while FIG. 1B depicts a cross section of the catheter.

FIG. 2 is a schematic of a set of catheters according to an aspect of the present invention, wherein each catheter comprises a unique solution pattern signature, which distinguishes each catheter from each other.

FIG. 3 is a photograph of the catheters inserted into brachytherapy catheters (i.e. applicators) implanted in a phantom tissue.

FIG. 4A is a T1-weighted fat-saturated gradient echo MRI image of the phantom tissue with applicators and the device in place.

FIG. 4B depicts a T1-weighted gradient echo 3T MRI without fat saturation of the phantom tissue with applicators and the device in place.

FIG. 5 is a maximum image intensity projection MRI volume rendering depicting the bright contrast of the inserted catheters using a T₁ weighted MRI sequence.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in brachytherapy applicator insertion process and the typical imaging systems needed to do treatment planning Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Brachytherapy Needle, Catheter, or Applicator” herein refers to any device that can be inserted into a patient and that can accept and hold a radiation source for the purpose of delivering radiation therapy.

“High Dose Rate (HDR) Remote Afterloading” herein refers to the process of delivering a radiation source with a mechanical apparatus designed to delivery radiation into a brachytherapy needle, catheter, or applicator. HDR brachytherapy is usually administered in less than one hour and it is typically given in multiple sessions

“Simulation Radiography” herein refers to (but is not limited to) the acquisition of images that permit one of several forms of treatment planning or that involves applicator position check within a patient.

“Dummy Ribbons” herein refers to devices that are inserted into brachytherapy applicators, and serve to mark the positions of the applicators within the patient and define the potential locations of the active radiation sources.

“Dwell Positions” herein refers to the location within the brachytherapy applicators where the active radiation sources may be positioned during treatment delivery

“Tip Dwell” herein refers to the most proximal position on the dummy ribbon. It is found at the end of the dummy ribbon and it may be specially marked to identify the end of the device.

“Brachytherapy Treatment Planning” is the process of using imaging and a treatment planning computer to calculate the dose distribution (dosimetry) in preparation for or description of brachytherapy.

“Dosimetry” is the process of calculating dose distributions within a patient or a phantom for purpose of radiation therapy

“Brachytherapy” herein refers to all forms of radiation where the radiation source is placed directly onto or into the treatment target.

“HDR, LDR, and PDR” herein refers, as defined in the introduction, to the method of brachytherapy radiation source deliver.

“Intracavitary or IC” herein refers to insertion of brachytherapy applicators into existing body cavities.

“Intraluminal or IL” herein refers to insertion of brachytherapy applicators in to body lumens (such as esophagus or bronchus)

“Interstitial or IS” herein refers to the insertion of brachytherapy catheters, needles, or other applicators directly into the target site (except natural body lumens accessible without tissue puncture)

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Disclosure

The present invention includes a catheter that can be filled with a solution of a contrast agent combined with saline, water or any other suitable aqueous solution. The solution of saline and contrast agent provides a bright contrast during medical imaging, for example under MRI, CT, and the like. The catheters of the present invention may be placed within another catheter, such as a brachytherapy applicator of any kind (including HDR, PDR, or LDR), that is positioned onto or within the treatment site. For example, the catheter of the present invention can be easily inserted into the brachytherapy applicator for visualization with MRI, and then subsequently removed so that administration of the radiation source and treatment delivery can be performed. As contemplated herein, the dimensions of the catheter and the exact placement of the contrast agent solution within the catheter can be adjusted so that the catheter can be accurately located relative to the tumor and normal organs during imaging. As demonstrated herein, the present invention provides bright contrast under MRI, which is preferred by clinicians as it significantly improves device conspicuity relative to surrounding tissues. It should be appreciated that the present invention may be used with any type of applicator, and therefore is suitable for use with all forms of brachytherapy, including HDR, LDR, PDR, IC, IL, and IS, without limitation. The catheter is not limited for use in conjunction with a brachytherapy applicator. Rather, the catheter may be positioned within any tube, channel, conduit, catheter, applicator, or the like that is positioned within the body for any application.

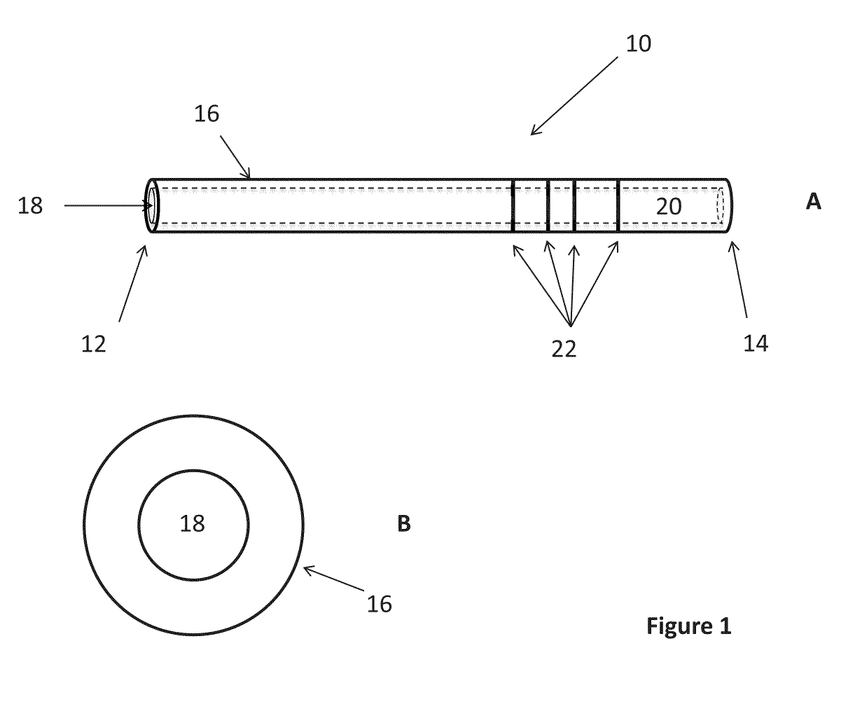

The present invention includes a device in the form of a catheter having sealed within, at least a portion of the lumen of the catheter, a solution containing one or more contrast agents and saline. For example, as illustrated in FIG. 1, the device (or catheter) 10 may be generally shaped as a cylindrical tube having a proximal end 12 and distal end 14. Catheter 10 has an exposed outer surface 16 surrounding an inner lumen 18. Catheter 10 may be fully or at least partially filled with a solution 20 that includes at least one contrast agent and saline. Catheter 10 may be capped at the proximal and distal ends 12 and 14 to keep solution 20 fully encapsulated within catheter 10, such that catheter 10 is free of any leakage. Optionally, catheter 10 may include additional features at the proximal end 12 for better handling and to facilitate insertion and removal of catheter 10 during use. Catheter 10 may be composed of a flexible material, such as, without limitation, a plastic or elastomeric polymer. Catheter 10 may be either re-useable or disposable. For example, if it is re-useable, then catheter 10 may be constructed such that it can be cleaned and/or sterilized according to industry standards.

Generally speaking, it should be appreciated that catheter 10 is sized and shaped to fit within the lumen of an implanted brachytherapy applicator, such that catheter 10 may be easily inserted and retracted from the brachytherapy applicator without disrupting the positioning of the brachytherapy applicator in the subject. As long as catheter 10 may readily be inserted and retracted from the brachytherapy applicator or other type of implanted cannula, catheter 10 is not limited to any particular dimensions or geometry. For example, a 6 F HDR applicator may have an inner diameter of about 4 F. Therefore, in one exemplary embodiment, catheter 10 may have an external diameter of 4 F or less. In another embodiment, the total length of catheter 10 should exceed the length of the HDR applicator so that it can be easily inserted and removed from the brachytherapy applicator. For example, and without limitation, the length of catheter 10 may be between 25-35 cm. In other embodiments, both shorter and longer catheters may be desired.

Because brachytherapy often involves the use of multiple brachytherapy applicators, it is important to uniquely identify each applicator during imaging so they can be uniquely identified during imaging or treatment planning, because each applicator and each HDR dwell position, for example, will have its own unique location and treatment time. It should be understood that the “tip dwell” is the most distally achievable location within the HDR applicator that can be reached by the actual radiation source during treatment. Accordingly, it should be clearly identified on the MRI images for computer treatment planning and dosimetry calculations. The tip dwell position is determined by the applicator lumen, applicator cap, and the physical dimensions of the radiation source and it must be calculable at least in part from the known geometry of catheter 10. Because catheter 10 is also sealed or capped at the distal end, the thickness of the sealing material or cap must be known precisely, as solution 20 will only be visible up to the proximal end of the sealing material, cap or distal end 14 of catheter 10.

Hence, catheter 10 may include specific markings 22 or segments along its length to aid in the identification of each catheter during an imaging exam. Markings 22 may be of any size, shape, or number that distinguish catheter 10 from all other catheters in use. In certain embodiments, catheter 10 comprises a dye that aids in the visualization of specific markings 22. For example, in one embodiment, markings 22 are regions of reduced contrast, as compared to the rest of catheter 10. In one embodiment, markings 22 comprise one or more non-contrast regions along the body of catheter 10. In certain embodiments markings 22 form a solution pattern signature, which allows for the “longitudinal coding” of catheter 10, thereby providing a clinician information on the identity of catheter 10 and corresponding brachytherapy applicator.

The solution pattern signature of catheter 10 may be formed using a variety of means. For example, in one embodiment, the solution pattern signature comprises a variation of the concentration of the contrast agent of solution 20 in a unique pattern along the length of catheter 10. In another embodiment, solution pattern signature is achieved by interrupting solution 20 so that it is voided or absent from unique spots along the length of catheter 10. For example, the solution pattern signature may comprise one or more interruptions of solution 20 along the length of catheter 10. For example, solution 20 can be interrupted with gel, bubbles, pinching or physical segregation/compartmentalization of the lumen 18 of catheter 10, so that no solution 20 is present in specific and unique locations. For example, in one embodiment, inner lumen 18 comprises a series of projections of the lumen wall that restrict solution 20, thereby forming markings 22 as regions of non-contrast. In one embodiment, lumen 18 is divided into one or more distinct compartments, where the compartments are separated by regions in which no solution 20 is present. The regions between the segments thus form markings 22 as regions of non-contrast. In this way, each catheter 10 may have a “signature,” or unique pattern of brightness and darkness along the length of catheter 10, such that the unique signature aids in identifying each catheter 10 in the visualization images.

Solution 20 may be formulated to provide bright or dark contrast to the portions of catheter 10 containing solution 20 when visualized by MRI. As mentioned previously, solution 20 is placed in at least a portion of lumen 18 of catheter 10 and sealed such that solution 20 does not leak out of catheter 10 or into any other remaining portion of lumen 18. In one embodiment, solution 20 fills the entire length of catheter 10. In another embodiment, solution 20 fills only a portion of catheter 10. It should be appreciated that in order for catheter 10 to have a “signature” as described above, solution 20 may have variable concentrations along the length of catheter 10, or it may have different concentrations in different segments or isolated compartments of catheter 10, as desired.

Solution 20 may include at least one contrast agent, and optionally multiple contrast agents. In one embodiment, the MRI contrast agent is a FDA approved contrast agent, such as Multihance® (gadobenate dimeglumine) or Magnevist® (gadopentetate dimeglumine), or any other FDA approved gadolinium-containing contrast agent. It should be appreciated that there is no limitation to the type or number of contrast agents suitable for inclusion into solution 20, provided that such contrast agent produces the desired visualization by MRI, including both bright contrast and dark contrast. In further embodiments, solution 20 also includes saline, water or any other aqueous solution suitable for diluting or mixing with the desired contrast agent. The amount and concentration of saline and the at least one contrast agent is not limited. For example, in one embodiment, the concentration of Multihance® may range from 0.62-1.19 mmol/L. In another embodiment, the concentration of Magnevist® may range from 0.62-2.22 mmol/L. These concentrations achieve a T1 in the range of 400-100 ms at 1.5T. As modifcations may be needed for other MRI field strengths, concentrations above or below these ranges may also be used. Solution 20 may also be compatible with commonly used antibacterial agents and other T1-shortening or T2-shortening agents. Preferably, the selected concentrations of contrast agent may provide contrast between catheter 10 and the subject's body tissues (including but not limited to tumor, fat, soft tissue, vascular structures, nerves, fascia, bone, visceral structures, and organ parenchyma) and saline.

In one embodiment, the present invention includes a system for visualizing one or more channels inserted into a tissue of the subject. The present system may be used for any application in which one or more tubes, channels, applicators, conduits, or the like, are positioned within the body, and where visualization and/or identification of one or more of the channels is required or beneficial. The system comprises one or more catheters 10, each catheter 10 having sealed within, at least a portion of lumen 18 of catheter 10, a solution 20 containing one or more contrast agents and saline. In one embodiment, each catheter 10 of the system comprises a set of markings 22, as described elsewhere herein, such that when visualized using MRI or other imaging modality, each catheter 10 is uniquely identifiable.

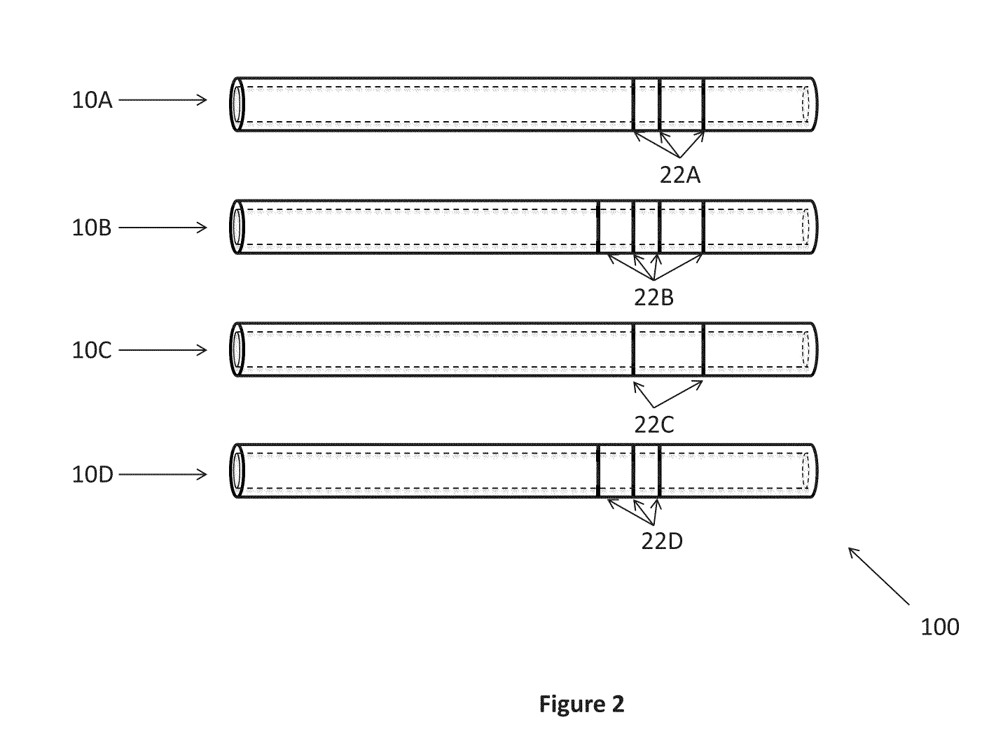

FIG. 2, depicts an exemplary system 100, comprising a set of catheters 10, labeled here as 10A through 10D. Each catheter 10 comprises a unique solution pattern signature, as defined by its markings 22 (labeled here as 22A through 22D) that allows identification by a clinician of a particular catheter 10. In certain embodiments, each catheter 10 is associated with, and inserted into, the one or more channels positioned within the body of a subject. Thus, identification of catheter 10, by way of the solution pattern signature, allows for visualization and identification of the positioned channel.

In one embodiment, the system is used for radiation treatment planning, where each catheter 10 is associated with a specific brachytherapy applicator. Thus, visualization and identification of each catheter 10 allows for effective visualization and identification of all brachytherapy applicators positioned within a treatment site.

In one embodiment, the system further comprises the one or more channels to be positioned within the body of a subject. For example, in one embodiment, the system comprises a one or more catheters 10 and one or more channels (i.e. applicators) to be positioned within the body, where each catheter 10 is configured to be inserted within one of the one or more channels to allow for visualization and identification of each channel.

In another embodiment, the present invention includes a method of visualizing the positioning of the brachytherapy applicator at a treatment site of a subject. The method includes positioning the distal end of an HDR applicator, for example, at the treatment site, inserting a catheter filled with a solution comprising a MRI contrast agent into the HDR applicator, examining the positioning of the catheter containing the solution via MRI, and retracting the catheter from the HDR applicator after the MRI exam. By confirming the positioning of the solution filled catheter during imaging, the radiation oncologist can determine the subsequent positioning of the delivered radiation source. Because treatment planning for HDR brachytherapy requires visualization of multiple treatment catheters, the catheter of the present invention can be used during each MRI exam. In some embodiments, catheter 10 may be filled with solution 20, sealed and inserted at the time of imaging. In other embodiments, catheter 10 may be pre-fabricated.

It should be appreciated that the present invention may be used in any application of brachytherapy, including and without limitation, applications for prostate, breast, head and neck, sarcoma, brain, eye, gynocological, anal, rectal and any other cancers (primary or metastatic). It should further be appreciated that the present invention can be used for any therapy that would benefit from MRI visualization of an implanted catheter or applicator, and particularly when such visualization includes insertion of the contrast agent filled catheter into an implanted applicator, cannula or other structure having a lumen.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

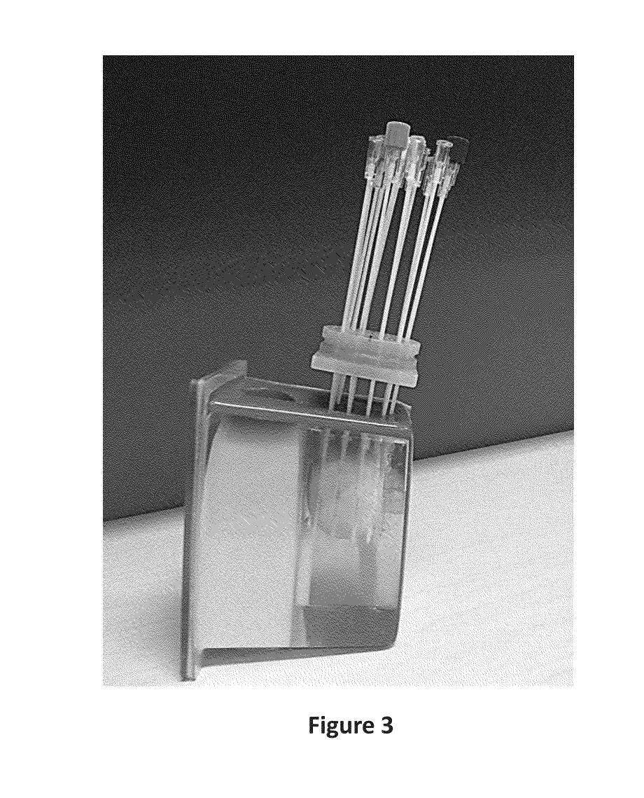

As illustrated in FIGS. 3-5, the present invention is demonstrated during in vitro MRI imaging. As shown in FIG. 3, the catheters of the present invention were filed with either Magnevist® or Multihance° and saline at concentrations of between 0.62-2.22 mmol/L. The catheters were inserted into brachytherapy applicators positioned in a phantom tissue (Supertech, Elkhart, Ind.). Images were acquired according to a clinical MRI prostate protocol.

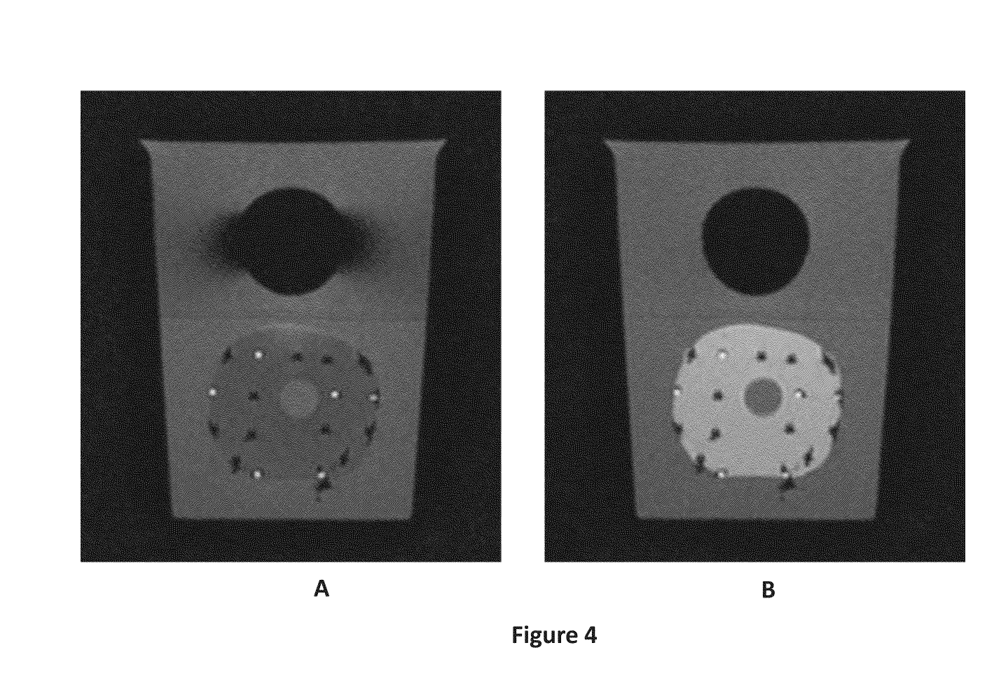

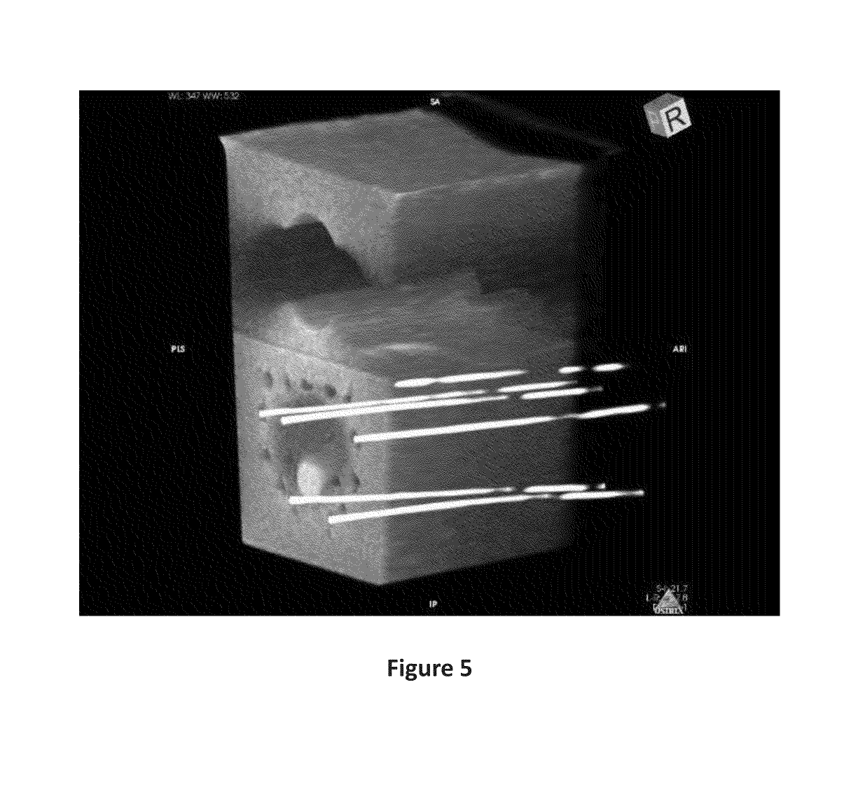

FIGS. 4A and 4B illustrate the imaging results of GRE with and without fat saturation respectively, acquired at 3T with the following imaging parameters: field of view=200×137 mm, acquisition matrix=256×204, resolution=0.6×0.6×3 mm, echo time=2.2 ms, repetition time=5.9 ms, bandwidth=625 Hz/px and flip angle=61°. As illustrated in FIG. 5, the catheters appear bright for a T₁ weighted sequence. Based on these results, the catheters of the present invention demonstrate a very simple, non-toxic and safe approach to enable the direct visualization of brachytherapy channels during treatment planning

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A catheter for providing contrast under magnetic resonance imaging (MRI), comprising:
an elongated tubular member having a proximal end, a distal end and lumen; and
a solution including saline and at least one contrast agent;
wherein the solution is sealed within at least a portion of the lumen to form a solution pattern signature.

2. The catheter of claim 1, wherein the contrast agent is a gadolinium-containing contrast agent.

3. The catheter of claim 1, wherein the contrast agent is gadobenate dimeglumine.

4. The catheter of claim 1, wherein the contrast agent is gadopentetate dimeglumine.

5. The catheter of claim 1, wherein the concentration of contrast agent is between 0.6-2.22 mmol/L.

6. The catheter of claim 1, wherein the solution pattern signature comprises a variation in contrast agent concentration along a length of the tubular member.

7. The catheter of claim 1, wherein the solution pattern signature comprises at least one interruption of the solution along a length of the tubular member, such that the solution is absent at the interruption.

8. The catheter of claim 7, wherein the interruption comprises a gel.

9. The catheter of claim 7, wherein the interruption comprises a bubble.

10. The catheter of claim 7, wherein the interruption comprises a pinched region of the tubular member.

11. The catheter of claim 7, wherein the interruption comprises a wall.

12. The catheter of claim 11, wherein the elongated tubular member includes multiple compartments, wherein at least two of the compartments are isolated from each other by a wall within the lumen.

13. A method of visualizing a channel positioned in the tissue of a subject, the method comprising:
positioning at least one channel in the tissue of a subject;
inserting into each channel a catheter having a solution including saline and at least one contrast agent sealed within the lumen of the catheter, such that the solution forms a pattern signature; and,
imaging the inserted catheter by magnetic resonance imaging (MRI).

14. The method of claim 13, wherein the channel is a high dose rate applicator (HDR) for brachytherapy.

15. The method of claim 13, further comprising constructing a treatment plan based on the visualization of each imaged catheter in each channel.

16. A system for visualizing a plurality of channels positioned in the tissue of a subject, the system comprising:
a plurality of catheters for providing contrast under magnetic resonance imaging (MRI), each catheter comprising an elongated tubular member having a proximal end, a distal end and lumen; and a solution including saline and at least one contrast agent; wherein the solution is sealed within at least a portion of the lumen to form a pattern signature;
wherein each catheter is uniquely identifiable by MR image and is associated with one of the one or more channels positioned in the tissue of the subject.

17. The system of claim 16, wherein the one or more channels positioned in the tissue of the subject comprises one or more brachytherapy applicators.

18. The system of claim 16, wherein the solution pattern signature comprises a variation in contrast agent concentration along a length of the tubular member.

19. The system of claim 16, wherein the solution pattern signature comprises at least one interruption of the solution along a length of the tubular member, such that the solution is absent at the interruption.

20. The system of claim 19, wherein the interruption comprises a gel.

21. The system of claim 19, wherein the interruption comprises a bubble.

22. The system of claim 19, wherein the interruption comprises a pinched region of the tubular member.

23. The system of claim 19, wherein the interruption comprises a wall.

24. The system of claim 23, wherein the elongated tubular member includes multiple compartments, wherein at least two of the compartments are isolated from each other by a wall within the lumen.
